(19) United States
(12) Patent Application Publication
(10) Pub. No.: US 2020/0376444 A1
(43) Pub. Date: Dec. 3, 2020

(54) Modified Filter Membrane and Method

(71) Applicant: BAYER HEAL THCARE LLC, New Jersey, NJ (US)

(72) Inventors: Shengjiang Liu, Lafayette, CA (US); Wensheng Wang, Pittsburgh, CA (US); Ashley Hesslein, San Francisco, CA (US)

(21) Appl. No.: 16/962,528

(22) PCT Filed: Feb. 15, 2019
(86) PCT No.: PCT/US2019/018216

§ 371 Date: Jul. 16, 2020

Related U.S. Application Data
(60) Provisional application No. 62/632,165, filed on Feb. 19, 2018.

(51) Int. Cl. B01D 71/10; A61L 2/00; B01D 67/00; B01D 69/02; B01D 71/02; B01D 69/08

(52) U.S. Cl. CPC B01D 71/10; A61L 2/0017; B01D 67/0088; B01D 69/02; B01D 2323/283; B01D 69/08; B01D 2325/02; B01D 2325/16; B01D 2325/20; B01D 71/022

(57) ABSTRACT

The embodiments provide a modified filter membrane for separating a crude solution of a biological product and a viral contaminant. The filter membrane has a cellulosed based porous surface, and at least one divalent metal ion bound to the cellulose based porous surface of the filter membrane to form a modified filter membrane cellulose based porous surface, wherein the modified cellulose based porous surface separates the crude solution by retaining a viral contaminant greater than 15 nm in diameter while allowing a biological product smaller than 15 nm in diameter to pass through. The embodiments also provide a method of filtering a crude solution of a biological product and a viral contaminant using a modified filter membrane by adding a divalent metal ion to a filter membrane porous surface to form a modified filter membrane porous surface with a pore size in the range of 1 to 15 nm in size, and filtering the crude solution of the biological product and the viral contaminant through the porous surface of the modified filter membrane, wherein the modified filter membrane retains the viral contaminant on the porous surface while allowing the biological product to pass through.

### BACKGROUND

Various filters and methods have been designed to separate biological molecules from impurities or contaminants. Size Exclusion based filtration is a desirable and effective technology for removing small particles and viruses derived from mammalian cells, plasma, animal/human tissue fluids or cell culture fluids from bioreactors of biological manufacturing processes. Current filtration technology can remove various contaminants, and virus particles above 15 nm of diameter. However, to date, no technology or filter exists for effective and robust removal of particles and tissue fluids below this size. For instance, small and non-enveloped viruses such as parvovirus and circovirus are very hard to remove from biological solutions. These small particles, viruses, and trace contaminants present issues in biologics manufacturing due to the fact that they can be amplified through cell culture and/or production manufacturing cycles. Further, it is very difficult to obtain a high and consistent recovery of biological product by a viral filtration filter and method when crude solutions or conditions vary and protein molecular size is large and protein concentration is very low. These and other problems have been addressed by the present embodiments.

### SUMMARY

The embodiments provide modified filter membranes for removal of small particles and other unknown or unidentified contaminants such as viruses present in a crude solution which needs to be separated from a biological product of interest.

The embodiments further provide a modified filter membrane for separating a crude solution of a biological product and a viral contaminant, comprising, a filter membrane having a cellulosed, based porous surface, and at least one divalent metal ion bound to the cellulose based porous surface of the filter membrane to form a modified filter membrane cellulose based porous surface, wherein the modified cellulose based porous surface separates the crude solution by retaining a viral contaminant greater than 15 nm in diameter while allowing a biological product smaller than 15 nm in diameter to pass through.

The embodiments also provide a method of filtering a crude solution of a biological product and a viral contaminant using a modified filter membrane, comprising adding a divalent metal ion to a filter membrane porous surface to form a modified filter membrane porous surface with a pore size in the range of 1 to 15 nm in size; and filtering the crude solution of the biological product and the viral contaminant through the porous surface of the modified filter membrane, wherein the modified filter membrane retains the viral contaminant on the porous surface while allowing the biological product to pass through.

Further embodiments can optionally comprise the addition of Tween 80 to further enhance the filter membrane separation compositions and methods and increase yields of biological products separated from crude solutions.

### BRIEF DESCRIPTION OF THE DRAWINGS

The skilled artisan will understand that the drawings, described below, are for illustration purposes only. The drawings are not intended to limit the scope of the present teachings or claims in any way.

FIG. 1 shows a filtration system using a virus filter membrane (e.g. Planova™ 20N). The filter load material is applied to the regenerated nitrocellulose hollow fiber membrane via a pressure source (e.g. compressed air or peristaltic pump). The filtrate is collected in a collection container.

FIG. 2 shows an enhanced section of a porous surface of an unmodified virus filter membrane.

FIG. 3 shows the method used in divalent metal ion enhanced viral filtration.

FIG. 4 shows various proposed mechanisms of the divalent metal ion cellulose based filters.

FIG. 5 shows enhanced removal of spiked PPV with virus filter membrane by the presence of CaCl₂) in load. (A) Filtration buffer is 2.0 mM Imidazole. 300 mM NaCl, 43 mM CaCl₂, pH=6.9-7.1. (B) Filtration buffer is 20 mM Imidazole, 375 mM NaCl, pH=6.9-7.1. Both A and B are performed under constant pressure (12-14 PSI) at ambient temperature.

FIG. 6 shows that removal of spiked PPV by a virus filter membrane is not significantly impacted by the presence of Tween 80 and the filtration temperature. (C) Filtration at ambient (15-26° C.) with 20 mM Imidazole, 300 mM NaCl, 43 mM CaCl₂), 50 ppm Tween 80, pH=6.9-7.1. (D) Filtration at 2-8° C. with 20 mM Imidazole, 300 mM NaCl, 43 mM CaCl₂, 50 ppm Tween 80, pH=6.9-7.1. (E) Filtration at ambient with 20 mM Imidazole, 300 mM NaCl, 43 mM CaCl₂, pH=6.9-7.1. (F) Filtration at 2-8° C. with 20 mM Imidazole, 300 mM NaCl, 43 mM CaCl₂, pH=6.9-7.1.

FIG. 7 shows the removal of spiked. PPV by virus filter membrane is not significantly impacted by the choice of different buffer systems. (G) Filtration at ambient with 20 mM Imidazole, 300 mM NaCl, 43 mM CaCl₂, pH=6.9-7.1. (H) Filtration at ambient with 20 mM Tris, 300 mM NaCl, 43 mM CaCl₂), pH=6.9-7.1. (I) Filtration at ambient with 50 mM Tris, 50 mM NaCl, pH=6.9-7.1. (J) Filtration at ambient temperature with 50 mM Citric Acid, 50 mM NaCl, pH=6.6-6.8.

FIG. 8 shows the enhancement of virus removal from PPV spiked IgG₂ antibody load (5.7-8.1 mg/mL in 50 mM Tris, 50 mM NaCl, pH 6.9-7.1) by virus filter membrane at different CaCl₂) concentrations and reverse of the enhancement by EDTA chelating agent.

FIG. 9 shows the enhancement of virus removal from PPV spiked IgG₁ antibody load (4.9-13.1 mg/mL in 50 mM Citric Acid, 50 mM NaCl, pH 6.6-6.8) by virus filter membrane at different CaCl₂) concentrations and no reverse of the enhancement by EGTA chelating agent.

FIG. 10 shows complete removal (to below limit of detection) of PPV from spiked rFVIII process intermediates (approximately 0.1 mg/mL in 20 mM Imidazole, 300 mM NaCl, 43 mM CaCl₂, 50 ppm Tween 80, pH=6.9-7.1) by virus filter membrane.

FIG. 11 shows the improvement of yield consistency of recombinant human Factor VIII by Tween 80 when using the virus filter membrane.

FIG. 12 shows the enhanced virus filter membrane capacity (VMax) by Tween 80 with application of recombinant human Factor VIII load.

### DETAILED DESCRIPTION

This disclosure provides compositions, including modified filter membranes for the removal of small particles and virus contaminants from a solution.

### Definitions

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

Whenever appropriate, terms used in the singular will also include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and are not limiting. The terms “such as,” “for example,” and “e.g.” also are not intended to be limiting. For example, the term “including” shall mean “including, but not limited to.”

As used herein, the terms “crude solution” or “crude load” refer generally to an unprocessed or unpurified solution or material which comprises one or more biological materials or molecules. Also present in this solution or material may be one or more contaminants which may or may not have been previously identified. For instance, a virus may be one type of contaminant present in a “crude solution”. It can also be anticipated that a “crude solution” also comprises other pathogens and contaminants which may be present or desirable to separate from a biological product of interest.

As used herein, the term “about” refers to +/−10% of the unit value provided.

As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term “substantially” is, therefore, used herein to capture the potential lack of completeness inherent in many biological and chemical phenomena.

Filter Systems:

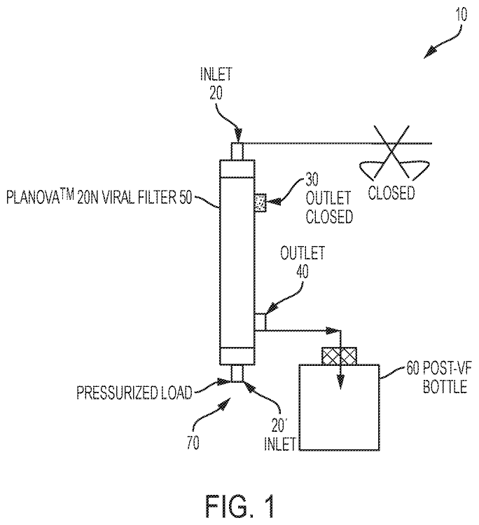

The described embodiments can be modified and changed in any number of ways. The described systems should in no way limit the scope of the possible applications and embodiments. Referring now to FIG. 1, the filtration system 10 of the present embodiments provides an inlet 20 or 20′, a closed outlet 30, an open outlet 40, a filter membrane such as a Pianova™ 20 N viral filter 50, and at least one collection container 60. The Filtration system 10 also has a pressurized load inlet 70 where a crude load can be loaded into the system and put under pressure to separate a biological product 22 from a contaminant (biological product 22 is not shown in FIG. 1).

### Filter Membrane:

The embodiments provide for the use of one or more filter membranes 50 that can be constructed or purchased. For instance, filter membrane 50 can have porous surfaces which comprise cellulose, regenerate cellulose, and/or nitrocellulose based materials or compositions. In addition, the filter membrane of the present embodiments can comprise an Asahi Planova® 20 N viral filter. Other types of filters known in the art can also be used with the present embodiments. It is important that the filter membrane 50 be capable of being modified to be effective for functionally removing contaminants of a defined size and shape from biological product 22 of interest that needs to be isolated, collected and/or purified. Further, the filter membrane 50 must be capable of binding one or more divalent metal ion to become modified filter membrane 50′ as further described below.

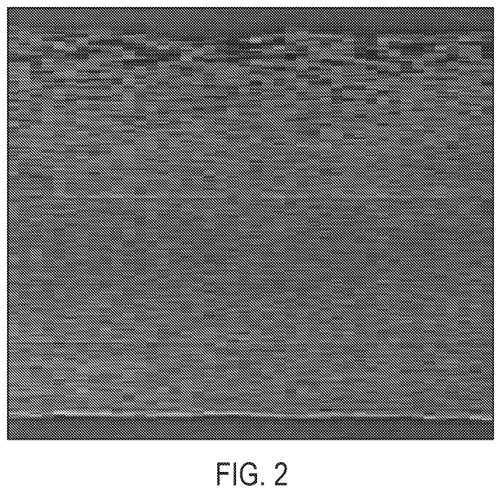

Regenerated cellulose based filter membranes with porous surfaces having unmodified nanometer pore size diameters are relatively high capacity and low cost. An enhanced view of the porous surface of regenerated cellulose membrane is shown in FIG. 2. The typical porous surface can comprise pore sizes that vary in shape and size. For instance, unmodified porous surface pore sizes can range in diameter of from 1 to 500 nm. Other possible pore sizes can also be possible. The porous surface and pore sizes can vary and generally are not effective in separating biological products from other proteins or contaminants. In many situations either the filter pore size is too large or the virus diameter is too small. In either case, it makes it particularly difficult to separate a biological product 22 from a virus or other contaminant.

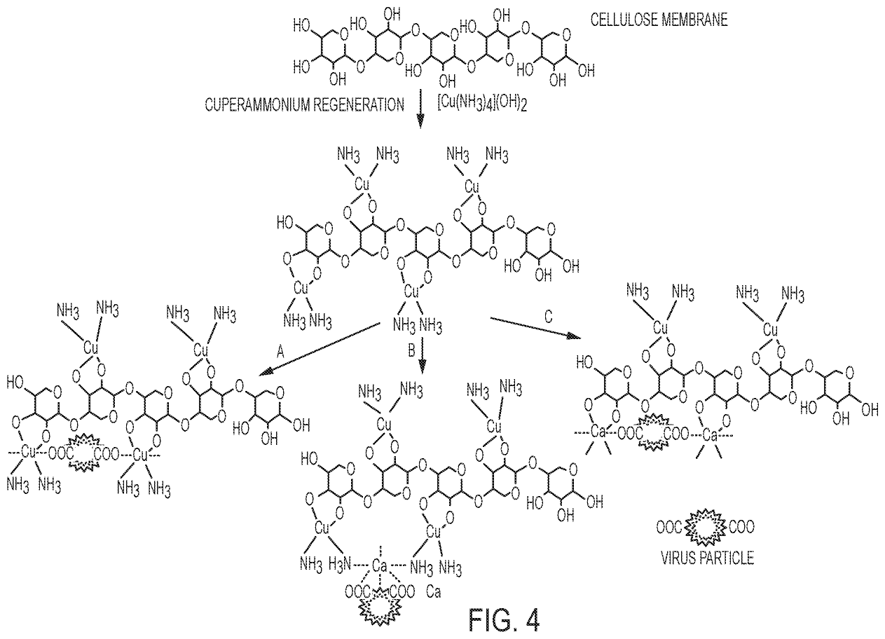

It is anticipated that the present embodiments can comprise various types of cellulose based filters. Cellulose is a linear polysaccharide of undefined numbers of glucose moieties. Cellulose is converted into cellulose derivatives (ethers and esters) and regenerated materials (fibers, films etc.) by classic viscose technology or cuprammonium process or N-methylmorpholine-N-oxide (NMMO) methods. The production of cellulose regenerated materials by cuprammonium processes introduces cuprammonia (H2N—Cu²⁺—NH2) complexed with hydroxyl groups of the cellulose via Cu²⁺ bridge. This structure theoretically can provide potential binding sites for calcium ions (See FIG. 4).

### Modified Filter Membrane:

Filter membranes need to be modified in order to make them effective for separating biological products from contaminants. The present embodiments require that bonding or attachment of one or more divalent ions to the filter membrane 50 to create modified filter membrane 50′ of the present embodiments. Divalent ions can be added to an assay solution and can comprise Cu²⁺, Ca²⁺, and Zn²⁺. Other similar divalent ions known in the art may be employed with the present embodiments.

### Proposed Mechanism:

It's important to the present embodiments that the modified filter membrane 50′ provides the functional ability of being able to separate a contaminant from a biological product 22 of interest from a crude load solution.

The mechanism of the viral retention enhancement by divalent metal ions such as Ca²⁺ ion is not well understood. One theory suggests that oxidation occurs during the cellulose regeneration process which causes the reducing ends of the cellulose chains to form carboxylic groups. Through dissociation of carboxyl groups the regenerated cellulose fibers can act as weak anion exchangers, thus all types of regenerated cellulose fibers (such as lyocell, viscose and modal fibers) show a distinct ability to bind Ca²⁺ ions. One possibility is that binding of Ca²⁺ ion with the carboxyl groups may apparently “narrow” the pores on the cellulose membrane and make it more difficult for virus to pass through. We further propose that other divalent ions (such as Cu²⁺) may have similar viral clearance enhancing effect for regenerated cellulose membranes especially some remaining Cu²⁺ is still present in the cellulose at low level when cuprammonium process was used for regeneration. A few proposed mechanisms suggest that Asp or Glu residues in viral contaminants bond with the modified filter membrane as shown FIG. 4. It is the divalent metal ions i.e. Cu²⁺ or Ca²⁺ that interacts with the hydroxyl groups of regenerated porous cellulose membrane and carboxyl groups of Asp or Glu in viral contaminants, resulting in virus binding to the filter membrane. In filtration tests of biological solutions, the size exclusion alone usually gives about 3-5 LRFs of parvovirus when regenerated cellulose filter is used. Once certain levels of divalent metal ions are used, additional 2-4 LRFs and/or complete removal of PPV can be achieved.

The modified filter membrane 50′ as mentioned can comprise cellulose or regenerated cellulose based porous surface. In the unmodified form, the pores of the filter membrane can typically comprise a pore diameter in a range of from 1 to 500 nm. Once a filter membrane has been modified, it can change its pore size. The pore size after modification is probably in a range of from 1 to 15 nm. It should be understood that these are only some general estimates and other possibilities and sizes are within the scope of the present embodiments.

### Virus Contaminants & Small Particles:

Further, it can also be anticipated that the modified filter membrane 50′ of the present embodiments can be used for separating a number of different types of biological products from contaminants. Contaminants can comprise any number of known or unknown biological materials, small particles and/or pathogens. For instance, viruses present one particularly difficult types of contaminant that needs to be carefully separated from biological products. Various types of viruses that relate to the present embodiments may be DNA and/or RNA viruses. For instance, a virus contaminant can comprise a DNA virus selected from the group consisting of Circoviridae, Adenoviridae, Parvoviridae, Papovaviridae, Herpesviridae, Poxiviridae, and Anelloviridae.

Further, the virus contaminants can also comprise an RNA virus selected from the group consisting of Picornaviridae, Caliciviridae, Reoviridae, Togaviridae, Arenaviridae, flaviviridae, Bunyaviridae. Orthomyxoviridae, Paramyxovirirdae, Filoviridae, Coronaviridae, Arteriviridae, Hepeviridase, and Retroviridae.

Other possible virus contaminants are also anticipated and known in the art. The present list should in no way limit the scope of the present embodiments.

### Biological Products:

The present embodiments and modified filter membrane 50′ can be employed to separate various contaminants from biological products 22. The biological products 22 which can be separated can comprise antibodies, proteins, peptides, ligands, and receptors. Various types of antibody classes can be separated from virus contaminants. For instance, the present embodiments are effective with IgG₁ and/or IgG₂ antibody loads. Further, certain proteins and protein fragments can comprise Factor VIII and associated fragments and/or truncated or deleted proteins and portions.

### Filtration Methods:

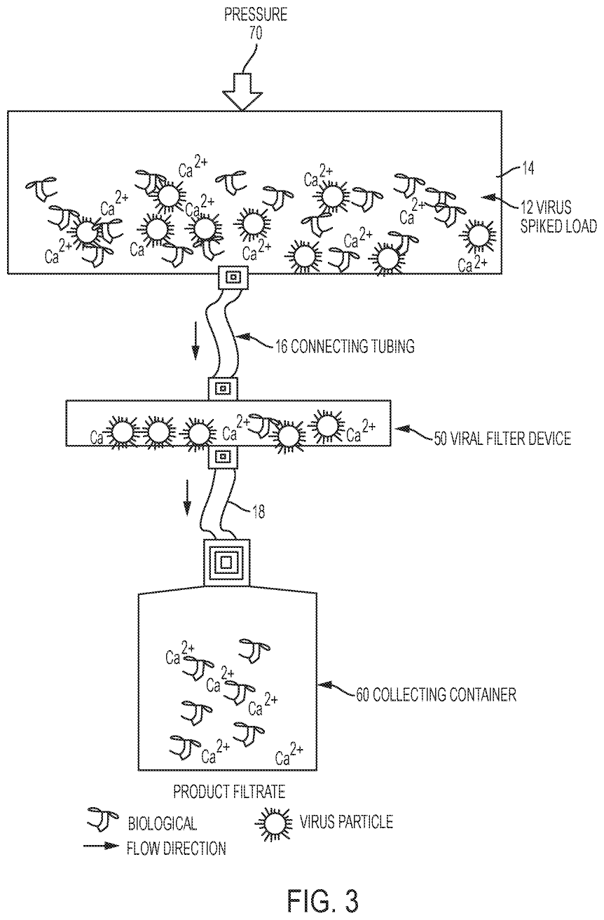

Having described the filtration system 10 and the filter membrane 50 and modified filter membrane 50′ compositions, it is now in order to describe how the present embodiments can be used to separate a contaminant from a biological product 22 of interest. Referring now to FIG. 3 the methods of the present embodiments will now be further described. The methods of the present embodiments begin by preparation of a crude load solution 12 (not shown) that is to be loaded into the filtration system 10. The crude load solution 12 comprises a biological product 22 of interest with one or more contaminants. Contaminants could be other proteins or biological materials and/or a virus. Also, present in the crude load solution 12 is an assay buffer that may comprise Tween 80, or one or more additional excipients, and at least one divalent metal ion such as Ca²⁺.

The crude load solution 12 is loaded at an inlet or inlet port 70 where is it can be under pressure and temporarily stored in collection chamber 14. The crude load solution 12 is then put under high pressure and passed through first connection tube 16 until it contacts filtration membrane 50. Any divalent metal ions present in the crude load solution and assay buffer bind to the porous surface of the filter membrane 50 changing it to a modified filter membrane 50′. The virus or contaminant is then bound to the modified filter membrane 50′ while a biological product 22 of interest may pass through to a second connection tube 18 which feeds into collection container 60. The final biological product 22 of interest can then be collected from collection container 60.

### EXAMPLES

### Example 1—Filtration Buffers

To achieve complete virus retention, the biological solutions comprise a neutral pH buffer, sodium chloride, at least one divalent metal ion i.e. Ca²⁺, Cu²⁺. Other components such as nonionic detergent may assist with the filtration process and removal of viral contaminants. The following buffers were generally used for filtration buffer matrix evaluation. Other buffers known in the art may also be employed.

20 mM Imidazole, 300 mM NaCl, 43 mM CaCl₂, pH=6.9-7.1;
20 mM Imidazole, 375 mM NaCl, pH=6.9-7.1;
20 mM Imidazole, 300 mM NaCl, 43 mM CaCl₂, 50 ppm Tween 80, pH=6.9-7.1;
20 mM Tris, 300 mM NaCl, 43 mM CaCl₂, pH=6.9-7.1;
50 mM Tris, 50 mM NaCl, pH=6.9-7.1;
50 mM Citric Acid, 50 mM NaCl, pH=6.6-6.8.

The filtration buffers were either prepared or purchased. All chemicals (e.g. imidazole, Tris, citric acid, sodium chloride, calcium chloride, Tween 80, EDTA and EGTA) were purchased from Fisher Scientific. Each buffer preparation was performed at ambient temperature by measuring appropriate amount of each component with a balance or cylinder, dissolving and mixing all constituents in purified water in a 500 mL or 1000 mL container with a solution volume close to the preparation target. The buffer pH was measured by a pH meter and adjusted to target pH range using either HCl or NaOH solutions. The final buffer solution was brought to the target volume by addition of purified water. Conductivity of each prepared buffer was also measured with a conductivity meter. Each prepared buffer was filtered through a 0.22 μm filter prior to use.

### Example 2—IgG₁ Filtration Load Material

Recombinant human IgG₁, 4.9-13.1 mg/mL in 50 mM Citric Acid, 50 mM NaCl, pH 6.6-6.8. This material was obtained from Bayer manufacturing facility. It was a process intermediate sample, i.e. eluate from a cation exchange column step in the purification process.

### Example 3—IgG₂ Filtration Load Material

Recombinant IgG₂, 5.7-8.1 mg/mL in 50 mM Tris, 50 mM NaCl, pH 6.9-7.1 was obtained elsewhere. It was a process intermediate sample, i.e. flow through from an anion exchange membrane adsorber step in a purification process.

### Example 4—Recombinant Fall Load Material

Recombinant human factor FIII (rFVIII), 0.1 mg/mL in 20 mM Imidazole, 300 mM NaCl, 43 mM CaCl₂, 0-100 ppm Tween 80, pH=6.9-7.1, This material was obtained elsewhere. It was a process intermediate sample, i.e. eluate from a cation exchange column step in a purification process.

### Example 5—Virus Stock

PPV (NADL-2 strain, ATCC # VR-742) stock was purchased from BioReliance (Rockville, Md.). The vendor certified virus titer was confirmed using 50% tissue culture infective dose (TCID₅₀) assay prior to use. The stock virus used for spiking the load material for the virus filter was approximately 10 log₁₀ TCID₅₀/mL.

### Example 6—Cell Line and Media

PK13 (ATCC # CRL-6489) cell line was purchased from ATCC. Dulbecco's Modified. Eagle's Medium (DMEM), fetal bovine serum (FBS), and Penicillin/Streptomycin (e.g. 100×) were purchased from Fisher Scientific. The culture growth medium and 2× assay medium used for PK13 cell culture and PPV TCID₅₀ assay were prepared by mixing the components of appropriate volumes in a sterile container followed by filtration through a 0.22 μm filter. The final prepared growth medium was DMFM, 10% FBS, 100 μg/mL Penicillin/Streptomycin. The final prepared 2× assay medium was DMFM, 4% FBS, 200 μg/mL Penicillin/Streptomycin.

### Assay Examples

### Example 1—Filtration Process

The filtration process through the virus filter membrane was driven by pressurized air or a peristaltic pump (e.g. Scilog FilterTec pump) set at constant pressure (See FIG. 1). Prior to use the filter was tested for membrane integrity using an air bubble point method known in the art. The viral filter was rinsed with water, and equilibrated with appropriate virus filtration buffer.

The biological load material was then applied to an unmodified virus filter at constant pressure of 12 to 14 PSI. The filter was further chased with virus filtration buffer after load completion. The effluent from the chase was collected and combined with the filtrate from the load step.

In each filtration run the amount of load and filtrate were measured by an analytical balance. The time duration for each step (load and chase) was also recorded for evaluation of average flow rate.

### Example 2—Virus Titration

PPV titration was performed using an end-point dilution assay, i.e. TCID₅₀ assay. PK13 cells were seeded at 2000-4000 cells per well in 96-well plates and incubated overnight at 37° C. with 4-6% CO₂ in humidified incubator according to Safety Laboratory standard operating procedures. The test and positive control samples were serially diluted (e.g. 1:3.2 serial dilution) in DMFM medium. Each dilution level was inoculated onto a corresponding column of 8 wells of the seeded PK13 cells (with spent medium removed, 100 μL inoculum per well), and was allowed to infect the cells in the above incubator for 1.5-2.5 hours. Finally 100 μL of 2× assay medium was added to each well and the assay plates were placed back into the incubator to allow continued infection and development of cytopathic effect (CPE) for 6-7 days. CPE was scored for each well corresponding to each sample dilution level and the virus titer was calculated using Spearman Kärber equation implemented in a controlled Microsoft Excel sheet.

### Example 3—Chromogenic Assay for rFVIII Activity

rFVIII activity was determined by a chromogenic assay method using a COATEST® FVIII kit (DiaPharma Cat. No. 824094). The FVIII standard curve dilution levels were from 1-10 mIU/mL according to European Pharmacopoeia 6.0 Section 2.7.4 (Assay of Human Coagulation Factor VIII). FVIII standard and controls used were Bayer internal products qualified against the World Health Organizaiton (WHO) FVIII standard. The teat samples were appropriately diluted so that the final FVIII concentration overlaps with the 1-10 mIU/mL range based on initial estimation. The chromogenic reactions and absorbance readout were performed according to the procedure described in the assay kit instructions and standard laboratory procedures. The rFVIII activity of the test samples were calculated from the linear regression fitted standard curve described above. The assay was repeated where the initial estimation of FVIII activity in the test samples failed to generate sample dilution levels that overlaped with the 1-10 mIU/mL range of the calibration curve.

### Example 4—Filtration of Virus Spiked Buffer Solutions

Filtration load was prepared by spiking PPV test virus (e.g. 1:100 spike ratio) into a buffer of interest as described above. The target amount of the load was applied to the virus filter membrane at 12-14 psi constant pressure with the filtrate collected (FIG. 1). Virus titers in the spiked load and filtrate samples were determined by PPV TCID₅₀ assay. Log₁₀ reduction factor (LRF), a measure of virus clearance capacity, was calculated as the log₁₀ difference between the total virus infectivity loaded onto the filter and the total virus infectivity in the filtrate.

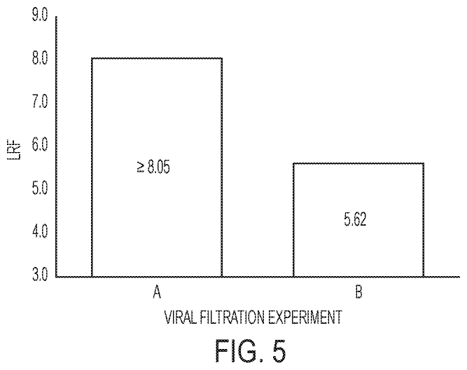

FIG. 5 shows that divalent Ca²⁺ ion (43 mM CaCl₂) in the load significantly enhanced PPV removal by the regenerated cellulose virus filter membrane. Virus titer in the filtrate was below the assay limit of detection with Ca²⁺ (panel A), indicating at least a two log₁₀ improvement of PPV clearance compared to no Ca²⁺ in the load (panel B).

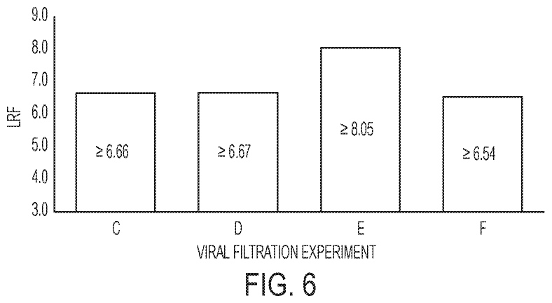

FIG. 6 shows that the enhanced virus clearance results by Ca²⁺ ion was not affected by the presence of Tween 80 (0 vs 50 ppm) and the filtration temperature (ambient vs 2-8° C.). Virus titer in the filtrate from all four filtration runs was below the assay limit of detection. The higher range shown in panel E was due to improved limit of detection using large sample volume TCID₅₀ assay.

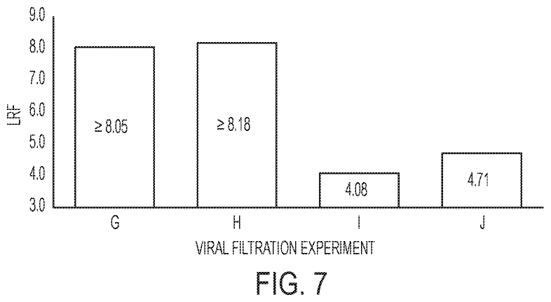

FIG. 7 shows that the enhanced virus clearance results by Ca²⁺ ion was not affected by the different buffer systems (20 mM Imidazole vs 20 mM Tris, or 50 mM Tris vs 50 mM Citric Acid). With Ca²⁺ the virus titer in filtrate was below assay limit of detection in imidazole (panel G) or Tris (panel H) buffer. Without Ca²⁺ the virus titer in filtrate was detected in both buffers (panels I and J), indicating at least, ≥1000-10000-fold or 3-4 log₁₀ reduction factor (LRF) viral clearance enhancement by Ca²⁺ ion.

### Example 5—Filtration of Virus Spiked IgG₂ Load

Load was prepared by spiking PPV test virus (e.g. 1:100 spike ratio) into a 5.7-8.1 mg/mL IgG₂ monoclonal antibody solution of purification process intermediate sample. The load was applied to the virus filter membrane at 12-14 psi constant pressure with the filtrate collected (FIG. 1). Virus titers in the spiked load and filtrate samples was determined by PPV TCID₅₀ assay. LRF was calculated as the log₁₀ difference between the total virus infectivity loaded onto the filter and the total virus infectivity in the filtrate.

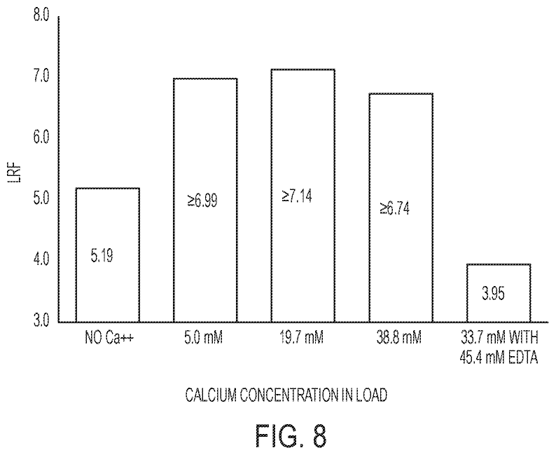

FIG. 8 shows the PPV clearance enhancement by Ca²⁺ ion at 5.0, 19.7 and 38.8 mM concentration levels, and that this enhanced was effectively reversed by the addition of 45.4 mM EDTA chelating agent in the load. Ca²⁺ ion already reached maximum effect at 5 mM and plateaued for viral clearance enhancement in the entire tested Ca²⁺ concentration range. With the addition of EDTA, a chelator for Ca²⁺, the viral clearance enhancement effect was no longer observed, indicating the observed enhancement effect is specifically due to the presence of Ca²⁺.

### Example 6—Filtration of Virus Spiked IgG₁ Load

Load was prepared by spiking PPV (e.g. 1:100 spike ratio) into a 4.9-13.1 mg/min IgG₁ monoclonal antibody solution of purification process intermediate sample. The load was applied to the virus filter membrane at 12-14 psi constant pressure with the filtrate collected (FIG. 1). Both the spiked load and filtrate samples were subjected to PPV TCID₅₀ assay to determine the virus titers. LRF was calculated as the log₁₀ difference between the total virus infectivity loaded onto the filter and the total virus infectivity in the filtrate.

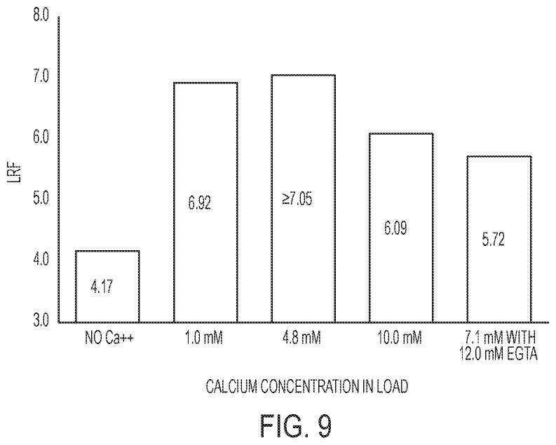

FIG. 9 shows the PPV clearance enhancement by Ca²⁺ ion at 1.0, 4.8 and 10.0 mM concentration levels, and that this enhancement was not significantly reversed by the addition of 12.0 mM EGTA chelating agent in the load (likely due to weak binding of EGTA to calcium ion). Ca²⁺ approached maximum effect at 1 mM and plateaued for viral clearance enhancement in the tested Ca²⁺ concentration range, indicating 2-3 log₁₀ viral clearance enhancement by Ca²⁺. The addition of EGTA (not EDTA), a specific chelator to Mg²⁺ instead of Ca²⁺, did not completely significantly reverse the viral clearance enhancement, reaffirming that the enhancement effect was specifically by Ca²⁺ ion.

### Example 7—Filtration of rFVIII Load

A non-spiked virus filtration load sample of Bayer rFVIII process intermediate was adjusted to include various concentration of Tween 80 (0-100 ppm), and was applied to the virus filter membrane at 12-14 psi constant pressure with the filtrate collected (FIG. 1). Both the load and filtrate samples were subjected to chromogenic assay to determine MIT activity. rFVIII yield was calculated as the percentage of total Fall activity in the filtrate compared to the total FVIII activity in the load (See Table 1).

TABLE 1
Virus Removal from Recombinant Human Factor VIII Process Intermediates
with a Regenerated Cellulose Filter in the Presence of CaCl₂

| | | | Log Reduction Factor for Model Virus (LRF) | | | |
|---|---|---|---|---|---|---|
| | | Apparent | PPV | Reo 3 | X-MuLV | PRV |
| Product | MW (kDa) | Size (nm) | 15-24 | 60-80 | 80-100 | 120-200 |
| rFVIII-WT | 300 | 14 | ≥6.13 ± 0.15 | ≥5.97 ± 0.15 | ≥5.12 ± 0.16 | ≥6.38 ± 0.19 |
| rFVIII-BDD | 224 | NT | ≥8.03 ± 0.28 | ≥5.82 ± 0.32 | ≥5.92 ± 0.25 | ≥4.30 ± 0.62 |

The LRF value in the Table is the average of three replicates (N=3) for each process condition. rFVIII-WT: recombinant human factor VIII (rFVIII) wild type and rFVIII-BDD:rFVIII binding domain deleted (BDD) submolecule of Fall. NT: The diameter of fFVIII-BDD was not measured but the size is approximately similar to that of rFVIII-WT.

Viral filtration load solutions where the manufacturing process intermediates containing recombinant wild type (wt) human factor VIII (rFVIII-WT) or recombinant human factor VIII binding domain deleted (BDD) (rFVIII-BDD) molecules were prepared in 20 mM imidazole, 300 mM NaCl, 43 mM CaCl₂, 50 ppm Tween 80, pH=6.9-7.1. The load samples were first spiked with porcine parvovirus (PPV), Reo 3 virus (Reo 3), xenotropic murine leukemia virus (X-MuLV) or porcine pseudorabies virus (PRY) individually and then filtered through a pre-filter of 0.45 μm filters (Corning cat. 430320 or equivalent) separately. The cuperammonia regenerated virus filters used were Planova® 20 N (0.001 m²), Asahi Kasei Medical Co., Cat. No. 20NZ-001 (9-1, Kanda Mitoshiro-cho, Chiyoda-ku, Tokyo, 101-8482 Japan). Viral filters were first flushed with the sample buffer and tested individually to ensure each was integral. Each virus spiked sample load was filtered through a Planova® 20 N (0.001 m²) viral filter. The virus titers in each virus spiked load and filtrates were determined by TCID₅₀ assay specifically designed for each virus. The virus removal results, log reduction factor (LRF) were calculated by subtracting virus titer in the filtrate from the titer of the load for each filtration experiment. For each virus spiked, three separate experiments were performed. The average value with 95% confidence interval was calculated using the three experimental results for each model virus. The results demonstrated that the regenerated cellulose viral filters (˜20 nm pore size) removed all the four mode viruses to the limit of detection (complete removal) using infectivity assays when the load solution contained. CaCl₂). The complete removal was not dependent on the morphology, size of the virus, nor the full length (rFVIII-wt) or BDD recombinant rFVIII (See Table 1).

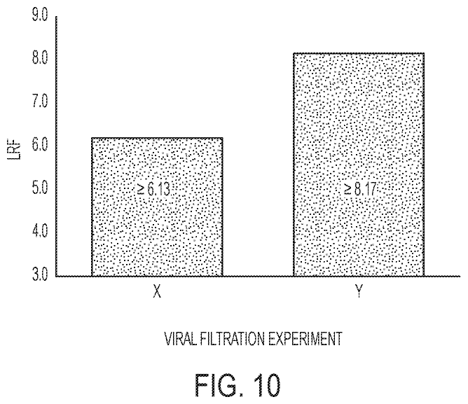

FIG. 10 shows complete removal (to below limit of detection) of PPV from spiked rFVIII process intermediates (approximately 0.1 mg/mL in 20 mM Imidazole, 300 mM NaCl, 43 mM CaCl₂, 50 ppm Tween 80, pH=6.9-7.1) by virus filter membrane.

Virus spiked Load was prepared by spiking PPV (e.g. 1:50 spike ratio) into a solution of rFVIII viral filtration load sample obtained from Bayer rFVIII manufacturing campaign process. The load was applied to the virus filter membrane at 12-16 psi constant pressure with the filtrate collected (FIG. 1). Both the spiked load and filtrate samples were subjected to PPV TCID₅₀ assay to determine the virus titers. LRF was calculated as the log₁₀ difference between the total virus infectivity loaded onto the filter and the total virus infectivity in the filtrate.

FIG. 10 shows complete PPV clearance results (to below the TCID₅₀ assay limit of detection) for two Bayer rFVIII products. Panel X is the average clearance result from three replicate filtration runs with load materials containing a full length rFVIII protein. Panel Y is the average result from six replicate filtration runs with load material containing B-domain deleted rFVIII protein. Both load materials contain approximately 0.1 mg/mL rFVIII in buffers with 43 mM CaCl₂ (the same buffer system as shown in FIG. 3, panel A). The relatively lower LRF range observed for in panel X was due to the factor that PPV stock virus of relatively lower titer was used in corresponding filtration studies. These results are consistent with the observation that the presence of Ca²⁺ ion in the load significantly enhances viral clearance by the filter membrane.

A regenerated cellulose hollow fiber membrane filter was used for separating a recombinant FVIII product with superior viral removal results and other biological production. During the development and optimization process the modified viral filtration membrane and filter were unexpectedly discovered. Further it was discovered that divalent metal ions such as calcium ion (Ca²⁺) can greatly enhance the parvovirus removal capability of regenerated cellulose based virus filter (Planova™ 20N, produced by Asahi Kasei Bioprocess, Inc.). The modified filter membrane and porous surface were effective with both protein and antibody crude loads. Porcine parvovirus (PPV) was used to evaluate viral contaminant removal by the modified virus filter membrane in our study since parvovirus is a small non-enveloped virus and represents a difficult virus for removal.

Porcine parvovirus is a non-enveloped single-stranded DNA virus in the family parvoviridae. This virus is usually selected as a non-specific model virus for evaluation of virus clearance by biological manufacturing process steps because it has a high tolerance to extreme chemical and physical environments. PPV virus particles are very small (15-24 nm), which makes it difficult to remove by size exclusion based viral filtration.

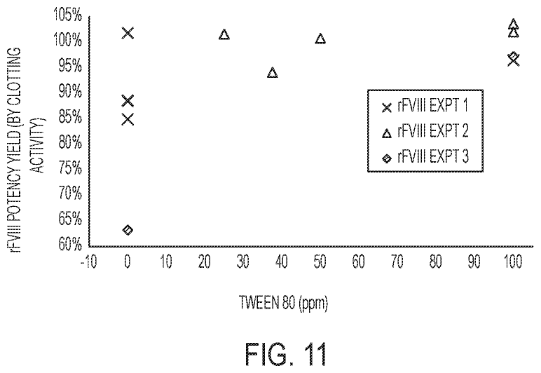

FIG. 11 shows that the presence of Tween 80 at 2:5-100 ppm corresponded to consistently high yield (94-104%), while the yield was less consistent (63-102%) without Tween 80.

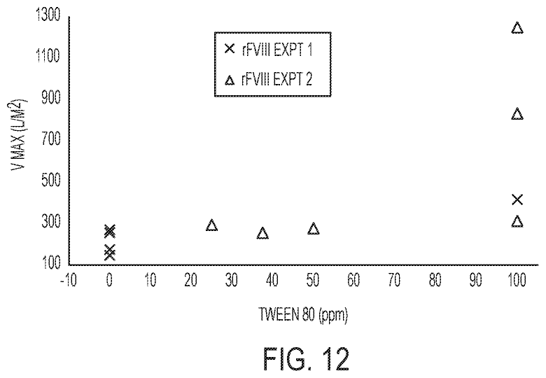

FIG. 12 shows that the capacity (VMax) of the virus filter membrane was higher in the presence of 25-100 ppm Tween 80 (256-1250 M/L²) than in the absence of Tween 80 (147-270 L/M²).

We claim:

1. A modified filter membrane for separating a crude solution of a biological product and a viral contaminant, comprising:
(a) a filter membrane having a cellulosed based porous surface; and
(b) at least one divalent metal ion bound to the cellulose based porous surface of the filter membrane to form a modified filter membrane cellulose based porous surface,
wherein the modified cellulose based porous surface separates the crude solution by retaining a viral contaminant greater than 15 nm in diameter while allowing a biological product smaller than 15 nm in diameter to pass through.

2. A modified filter membrane as recited in claim 1, wherein the divalent metal ion is selected from the group consisting of Cu²⁺, Ca²⁺, and Zn²⁺.

3. A modified filter membrane as recited in claim 2, wherein the virus contaminant comprises a non-enveloped virus particle or virus.

4. A modified filter membrane as recited in claim 3, wherein the virus contaminant comprises a parvovirus or circovirus in the range of 15-25 nm.

5. A modified filter membrane as recited in claim 3, wherein the virus contaminant further comprises a DNA virus selected from the group consisting of Circoviridae, Adenoviridae, Parvoviridae, Papovaviridae, Herpesviridae, Poxiviridae, and Anelloviridae.

6. A modified filter membrane as recited in claim 3, wherein the virus contaminant further comprises an RNA virus selected from the group consisting of Picornaviridae, Caliciviridae, Reoviridae, Togaviridae, Arenaviridae, Bunyaviridae, Orthomyxoviridae, Paramyxovirirdae, Filoviridae, Coronaviridae, Arteriviridae, Hepeviridase, and Retroviridae.

7. A modified filter membrane as recited in claim 1, wherein the crude solution comprises a protein solution.

8. A modified filter membrane as recited in claim 1, wherein the divalent metal ion derives from a calcium chloride compound.

9. A modified filter membrane as recited in claim 1, wherein the filter membrane comprises an Asahi Planova® 20 N viral filter.

10. A modified filter membrane as recited in claim 1, wherein the modified cellulose base porous surface comprises a pore size in the range of from 1 to 30 nm.

11. A modified filter membrane as recited in claim 1, wherein the cellulose based porous surface comprises a pore size in the range of 1 to 500 nm.

12. A method of filtering a crude solution of a biological product and a viral contaminant using a modified filter membrane, comprising:
(a) adding a divalent metal ion to a filter membrane porous surface to form a modified filter membrane porous surface with a pore size in the range of 1 to 15 nm in size; and
(b) filtering the crude solution of the biological product and the viral contaminant through the porous surface of the modified filter membrane, wherein the modified filter membrane retains the viral contaminant on the porous surface while allowing the biological product to pass through.

13. A method as recited in claim 12, wherein the filter membrane comprises a cellulose or regenerated cellulose material.

14. A method as recited in claim 12, wherein the filter membrane comprises an Asahi Planova® 20 N viral filter.

15. A method as recited in claim 12, wherein the crude solution further comprises a nonionic detergent selected from the group consisting of polysorbate 80, Tween 80, Tween 20, N-methylglucamides (MEGA) derivatives, Triton X and derivatives.

16. A method as recited in 12, wherein porous surface comprises a pore size of from 1 to 500 nm.

17. A method as recited in claim 12, wherein the modified porous surface comprises a pore size of from 1 to 15 nm in size.
